(19) United States
(12) Patent Application Publication
(10) Pub. No.: US 2014/0018627 A1
(43) Pub. Date: Jan. 16, 2014

(54) ENDOSCOPY APPARATUS HAVING HIGH DEGREE OF MOTION FREEDOM AND OPERATING METHOD THEREOF

(71) Applicant: Crystalvue Medical Corporation, Gueishan (TW)

(72) Inventors: Chung-Cheng CHOU, Lujhu Township (TW); William WANG, Taoyuan City (TW)

(73) Assignee: CRYSTALVUE MEDICAL CORPORATION, Gueishan (TW)

(21) Appl. No.: 14/031,405

(22) Filed: Sep. 19, 2013

Related U.S. Application Data
(62) Division of application No. 13/157,832, filed on Jun. 10, 2011.

(30) Foreign Application Priority Data
Jun. 11, 2010 (TW) 099119083

(51) Int. Cl. A61B 1/008

(52) U.S. Cl. CPC A61B 1/008
USPC 600/141

(57) ABSTRACT

An endoscopy apparatus having high degree of motion freedom and the operating method thereof are disclosed. The endoscopy apparatus having high degree of motion freedom includes a multi-stage endoscopy module and a control module. The multi-stage endoscopy module includes at least a first endoscopy unit and a second endoscopy unit. The first endoscopy unit and the second endoscopy unit can provide a first bending and a second bending respectively. The second bending is larger than the first bending. When the multi-stage endoscopy module moves to a region near a target observing position, the control module will control the second endoscopy unit to generate slight deformation to observe a real-time state of the target observing position.

### CROSS REFERENCE TO RELATED APPLICATIONS

The invention is a divisional application of U.S. patent application Ser. No. 13/157,832, filed on Jun. 10, 2011, which is incorporated by reference in its entirety, and which claims priority from Taiwan Application Serial No. 099119083, filed Jun. 11, 2010.

### BACKGROUND OF THE INVENTION

1. Field of the Invention

The invention relates to an endoscopy apparatus, in particular, to an endoscopy apparatus having high degree of motion freedom and operating method thereof.

2. Description of the Prior Art

In recent years, with the continuous progress of medical technology, the medical equipment is also developed toward the direction of innovation. Therefore, more and more advanced medical equipments have been widely applied in clinical diagnosis and treatment. For example, an endoscopy is usually used to observe the organs in the body of the patient.

In general, the endoscopy is mainly used to observe, test, or even treat the organs in the body of the patient. Its main principle is to use a thin optical lens extending into the human body and transmit the enlarged image to the monitor to show the structure inside the human body. And, the endoscopy can use various thin apparatuses to perform treatments avoiding the nerve and vascular under minimum injury. Because the endoscopy causes smaller would and less bleeding, and the recovery period is shorter, the patient can rapidly recover to the ordinary life.

In practical applications, because the positions and shapes of the organs in human body are not the same, there are a lot of researches of the driving mechanism or degree of motion freedom of the endoscopy. Please refer to the following US patents: U.S. Pat. No. 4,054,128, U.S. Pat. No. 6,800,056, and U.S. Pat. No. 7,637,905. FIG. 1, FIG. 2, and FIG. 3 are representative figures of the above-mentioned three US patents respectively.

According to the technologies disclosed by the above-mentioned three US patents, the endoscopies in these patents may be applied to different body parts and have different purposes, but they all use the same design of multi-link type. However, in practical applications, when the endoscopies are used to perform detail test, sampling, or treatment, no matter what kind of design has disadvantages of operating difficulties, therefore, the experienced medical personnel is clinically necessary to fully achieve the effect of test.

Therefore, the invention provides an endoscopy apparatus having high degree of motion freedom and operating method thereof to solve the above-mentioned problems.

### SUMMARY OF THE INVENTION

A scope of the invention is to provide an endoscopy apparatus having high degree of motion freedom. Because the endoscopy apparatus has an endoscopy design of multi-stage type different from the conventional endoscopy design of multi-link type in prior arts, the endoscopy apparatus of the invention can be used to perform more detail test, sampling, or treatment on the organs in patient's body. Therefore, the quality of medical care can be enhanced and the health and the right of the patient can be also ensured accordingly.

A first embodiment of the invention is an endoscopy apparatus having high degree of motion freedom. In fact, the endoscopy apparatus having high degree of motion freedom is used to observe, test, or even treat the organs in the body of the patient.

In this embodiment, the endoscopy apparatus having high degree of motion freedom includes a multi-stage endoscopy module and a control module. The multi-stage endoscopy module includes at least a first endoscopy unit and a second endoscopy unit. The first endoscopy unit and the second endoscopy unit can provide a first bending and a second bending respectively. The second bending is larger than the first bending. When the multi-stage endoscopy module moves to a region near a target observing position, the control module will control the second endoscopy unit to generate slight deformation to observe a real-time state of the target observing position.

In practical applications, the multi-stage endoscopy module can be designed in different types. For example, an end of the multi-stage endoscopy module can be composed of the second endoscopy unit, and the other parts of the multi-stage endoscopy module except the end are composed of the first endoscopy unit, the multi-stage endoscopy module controls the movement or rotation of the second endoscopy unit on the end to observe the real-time state of the target observing position.

In addition, the second endoscopy unit can be encapsulated within the first endoscopy unit, when the multi-stage endoscopy module moves to the region near the target observing position, the control module controls the second endoscopy unit extending out of the first endoscopy unit, or controls the first endoscopy unit to shrink to expose the second endoscopy unit out of the first endoscopy unit.

Another scope of the invention is to provide a method of operating an endoscopy apparatus having high degree of motion freedom. The method is used to operate the endoscopy apparatus having a multi-stage endoscopy design to perform more detail test, sampling, or treatment on the organs in patient's body.

A second embodiment of the invention is a method of operating an endoscopy apparatus having high degree of motion freedom. In fact, the endoscopy apparatus having high degree of motion freedom includes a multi-stage endoscopy module and a control module. The multi-stage endoscopy module includes at least a first endoscopy unit and a second endoscopy unit. The first endoscopy unit and the second endoscopy unit provide a first bending and a second bending respectively, wherein the second bending is larger than the first bending.

In this embodiment, the method includes the steps of: (a) the multi-stage endoscopy module moving to a region near a target observing position; (b) the control module controlling the second endoscopy unit to generate slight deformation; (c) the second endoscopy unit observing a real-time state of the target observing position.

Compared to the prior arts, the invention discloses the endoscopy apparatus having high degree of motion freedom and operating method thereof not only can maintain the advantage of the conventional endoscopy to smoothly move and test in human body or organ, but also can control the soft endoscopy unit to generate slight deformation to perform a detail test or sampling on the target observing position through a movement or rotation.

Therefore, the endoscopy apparatus having high degree of motion freedom and the operating method thereof disclosed by the invention can overcome the problems that the current endoscopy encountered clinically, and can be used to perform more detail test, sampling, or treatment on the organs in patient's body. Therefore, the quality of medical care can be enhanced and the health and the right of the patient can be also ensured accordingly.

The advantage and spirit of the invention may be understood by the following detailed descriptions together with the appended drawings.

### BRIEF DESCRIPTION OF THE APPENDED DRAWINGS

FIG. 1, FIG. 2, and FIG. 3 illustrate representative figures of the above-mentioned three US patents in prior art respectively.

FIG. 4 illustrates the multi-stage endoscopy module in the first embodiment of the invention.

FIG. 5A, FIG. 5B, and FIG. 5C illustrate another example of the multi-stage endoscopy module of the invention.

FIG. 6A and FIG. 6B illustrate another example of the multi-stage endoscopy module of the invention.

FIG. 7A, FIG. 7B, and FIG. 7C illustrate another example of the multi-stage endoscopy module of the invention.

FIG. 8 illustrates a flowchart of the endoscopy apparatus having high degree of motion freedom operating method in the second embodiment of the invention.

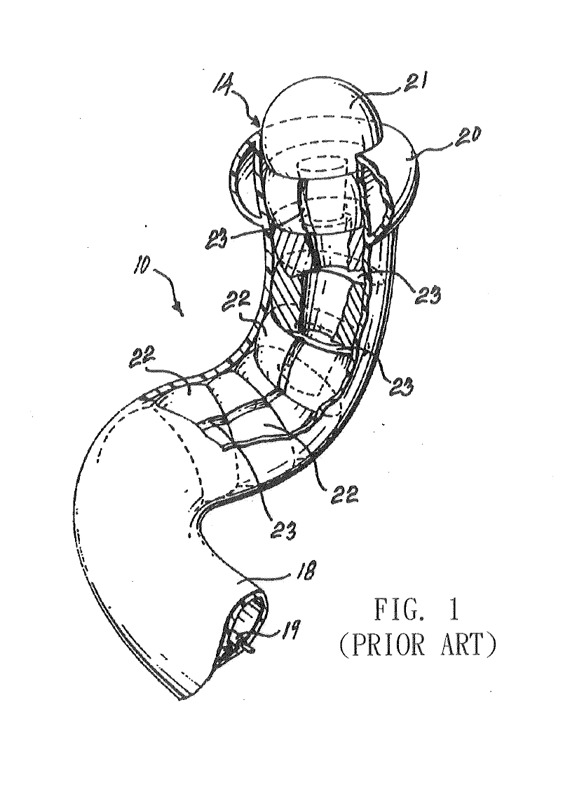

### DETAILED DESCRIPTION OF THE INVENTION

A first embodiment of the invention is an endoscopy apparatus having high degree of motion freedom. In fact, the endoscopy apparatus having high degree of motion freedom can be used to perform the test, sampling, or treatment on the organs in patient's body, but not limited to this. Because the endoscopy apparatus has an endoscopy design of multi-stage type different from the conventional endoscopy design of multi-link type in prior arts, the endoscopy apparatus of the invention can be used to perform more detail test, sampling, or treatment on the organs in patient's body.

In this embodiment, the endoscopy apparatus having high degree of motion freedom includes a multi-stage endoscopy module and a control module. The multi-stage endoscopy module includes at least a first endoscopy unit and a second endoscopy unit. That is to say, the multi-stage endoscopy module can include two or more endoscopy units, and the number of the endoscopy units can be adjusted based on practical needs, it is not limited by the two endoscopy units in this embodiment.

It should be noticed that the first endoscopy unit and the second endoscopy unit can provide a first bending and a second bending respectively, wherein the second bending is larger than the first bending. That is to say, because the material of the second endoscopy unit is softer than that of the first endoscopy unit, the second endoscopy unit will generate deformation more easily than the first endoscopy unit.

When the multi-stage endoscopy module moves to a region near a target observing position (e.g., stomach in a human body), the control module will control the second endoscopy unit to generate slight deformation (e.g., movement or rotation) to approach the target observing position to observe a real-time state of the target observing position in detail. For example, the real-time state can be whether the stomach wall is perforated or has other lesions, but not limited to this case.

The multi-stage endoscopy module of the invention can be designed in different types; therefore, various design types of multi-stage endoscopy modules will be introduced respectively as follows.

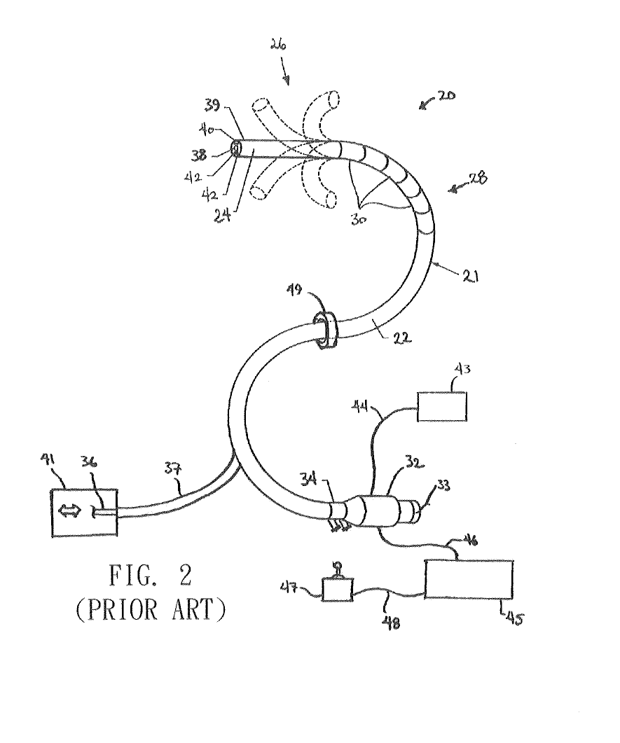

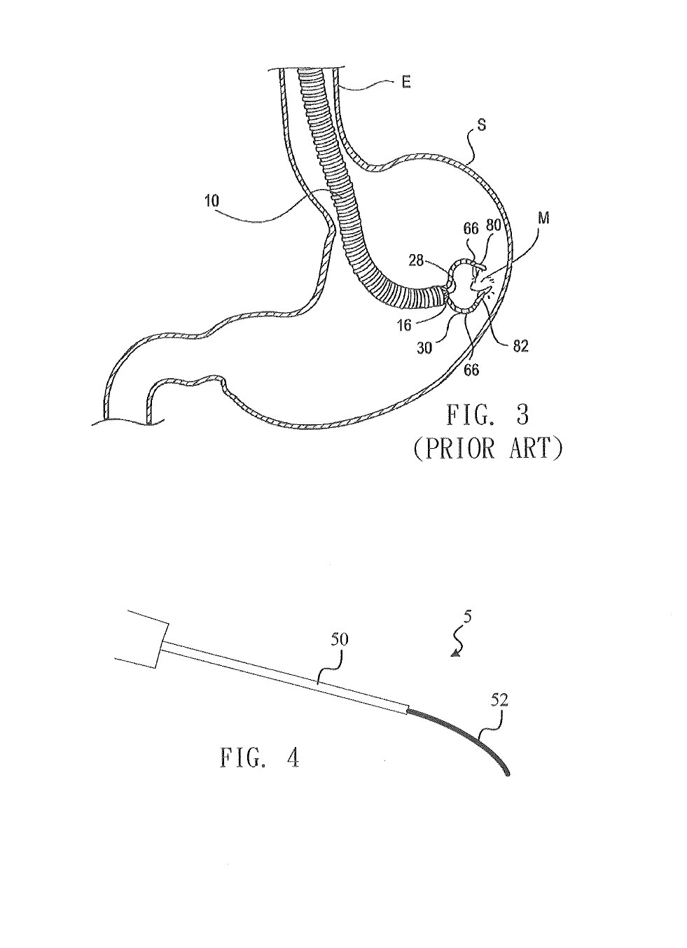

Please refer to FIG. 4. FIG. 4 illustrates an example of the multi-stage endoscopy module of the invention. As shown in FIG. 4, the multi-stage endoscopy module 5 includes a first endoscopy unit 50 and a second endoscopy unit 52. Wherein, the material of the first endoscopy unit 50 is harder, and the material of the second endoscopy unit 52 is softer. An end of the multi-stage endoscopy module 5 is composed of the second endoscopy unit 52, and the other parts of the multi-stage endoscopy module 5 except the end are composed of the first endoscopy unit 50. Therefore, the multi-stage endoscopy module 5 controls the movement or rotation of the second endoscopy unit 52 on the end to observe the real-time state of a certain target observing position.

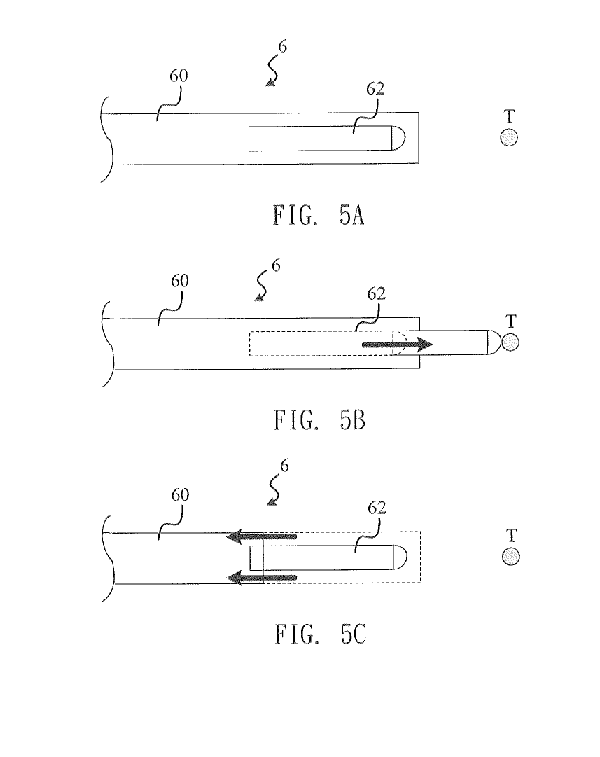

Please refer to FIG. 5A, FIG. 5B, and FIG. 5C. FIG. 5A, FIG. 5B, and FIG. 5C illustrate another example of the multi-stage endoscopy module of the invention. As shown in FIG. 5A, in the multi-stage endoscopy module 6, the second endoscopy unit 62 is encapsulated within the first endoscopy unit 60. Since the material of the first endoscopy unit 60 is hard, the multi-stage endoscopy module 6 can smoothly move in organs of the human body, and the first endoscopy unit 60 will not form an obstruction to movement due to the soft material.

As shown in FIG. 5B, when the multi-stage endoscopy module 6 moves to the region near the target observing position T, the control module (not shown in the figures) of the endoscopy apparatus controls the second endoscopy unit 62 extending out of the first endoscopy unit 60 and controls the second endoscopy unit 62 to generate slight deformation to observe the real-time state of the target observing position T through the movement or rotation of the second endoscopy unit 62.

Next, please refer to FIG. 5C. The most difference between FIG. 5C and FIG. 5B is that FIG. 5B shows a way of controlling the second endoscopy unit 62 automatically extending out of the first endoscopy unit 60, but FIG. 5C shows a way of controlling the first endoscopy unit 60 to shrink to expose the second endoscopy unit 62 out of the first endoscopy unit 60.

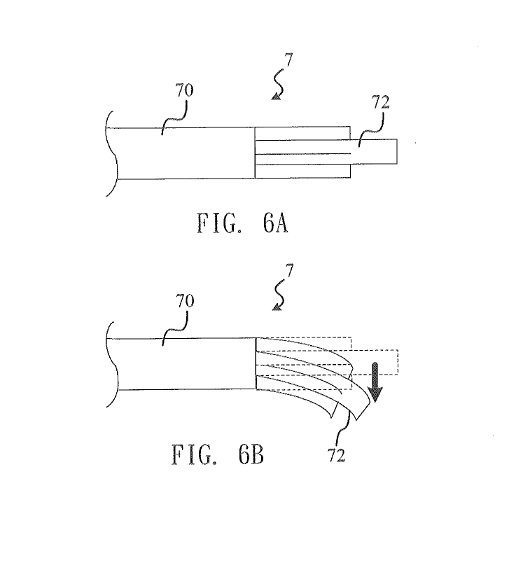

In addition, as shown in FIG. 6A and FIG. 6B, the connecting mechanism between the first endoscopy unit 70 and the second endoscopy unit 72 of the multi-stage endoscopy module 7 can be used as a driving mechanism for providing deformations. In FIG. 6A, the second endoscopy unit 72 provides the attaching and fixing effect through the connecting mechanism to avoid the unexpected deflection caused due to the multi-stage endoscopy module 7 which is too soft. In FIG. 6B, when the multi-stage endoscopy module 7 approaches the target observing position, this connecting mechanism can provide the driving deformation of the second endoscopy unit 72 to reach the purpose of observation in detail.

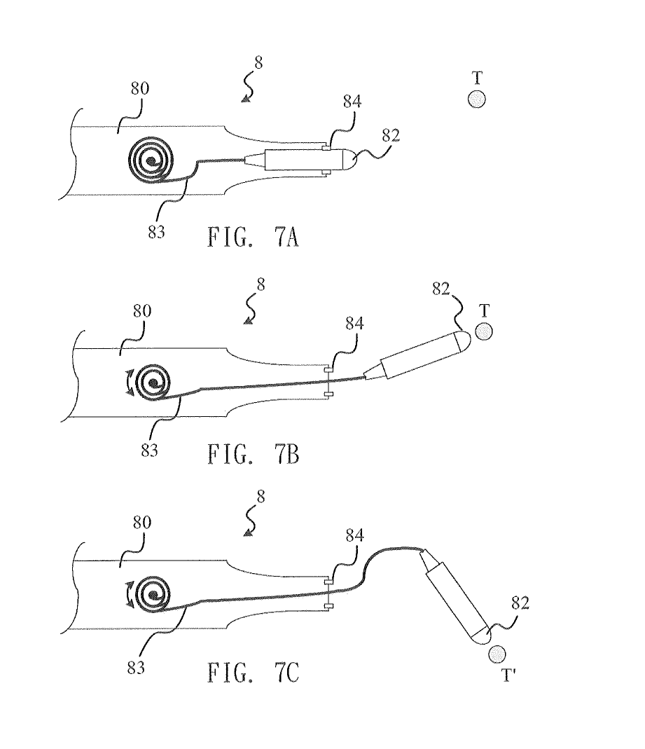

Please refer to FIG. 7A, FIG. 7B, and FIG. 7C. FIG. 7A, FIG. 7B, and FIG. 7C illustrate another example of the multi-stage endoscopy module of the invention. As shown in FIG. 7A, the multi-stage endoscopy module 8 includes a first endoscopy unit 80 and a second endoscopy unit 82. Wherein, the second endoscopy unit 82 is coupled to a surrounding pipeline 83 and fixed in the first endoscopy unit 80 through a fixing ring 84.

As shown in FIG. 7B, when the multi-stage endoscopy module 8 moves to the region near the target observing position T, the fixing ring 84 will be released to make the second endoscopy unit 82 extending out of the first endoscopy unit 80 and then the second endoscopy unit 82 generates slight deformation, therefore, the multi-stage endoscopy module 8 can move the second endoscopy unit 82 to observe the real-time state of the target observing position T. In addition, as shown in FIG. 7C, the multi-stage endoscopy module 8 can also rotate the second endoscopy unit 82 to observe the real-time state of another target observing position T′.

A second embodiment of the invention is a method of operating an endoscopy apparatus having high degree of motion freedom. In this embodiment, the endoscopy apparatus having high degree of motion freedom includes a multi-stage endoscopy module and a control module. The multi-stage endoscopy module includes at least a first endoscopy unit and a second endoscopy unit. The first endoscopy unit and the second endoscopy unit provide a first bending and a second bending respectively, wherein the second bending is larger than the first bending. That is to say, because the material of the second endoscopy unit is softer than that of the first endoscopy unit, the second endoscopy unit will generate deformation more easily than the first endoscopy unit.

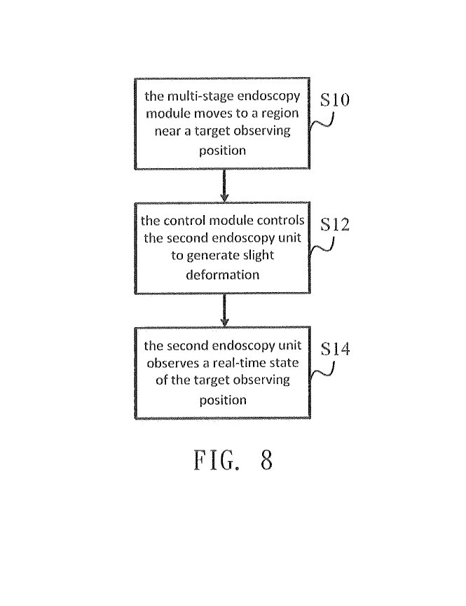

Please refer to FIG. 8. FIG. 8 illustrates a flowchart of the endoscopy apparatus having high degree of motion freedom operating method. As shown in FIG. 8, the method includes the following steps. At first, in the step S10, the multi-stage endoscopy module moves to a region near a target observing position. In fact, the target observing position can be any organ in any organisms, such as an intestinal or stomach, but not limited to this case. It should be noticed that the multi-stage endoscopy module smoothly moves to the region near the target observing position through its harder first endoscopy unit.

Then, in the step S12, the control module controls the second endoscopy unit to generate slight deformation. In fact, the slight deformation generated by the second endoscopy unit can be a movement, a rotation, or any other types of deformation without any limitations. In the step S14, the second endoscopy unit observes a real-time state of the target observing position. For example, the real-time state can be whether the stomach wall is perforated or has other lesions, but not limited to this case.

In an embodiment, an end of the multi-stage endoscopy module is composed of the second endoscopy unit, and the other parts of the multi-stage endoscopy module except the end are composed of the first endoscopy unit. Therefore, the multi-stage endoscopy module controls the movement or rotation of the second endoscopy unit on the end to observe the real-time state of the target observing position.

In another embodiment, in the multi-stage endoscopy module, the second endoscopy unit is encapsulated within the first endoscopy unit. When the multi-stage endoscopy module moves to the region near the target observing position, the control module controls the second endoscopy unit extending out of the first endoscopy unit or controls the first endoscopy unit to shrink to expose the second endoscopy unit out of the first endoscopy unit, and controls the second endoscopy unit to generate slight deformation to observe the real-time state of the target observing position through the movement or rotation of the second endoscopy unit.

In another embodiment, in the multi-stage endoscopy module, the second endoscopy unit is a surrounding pipeline and fixed in the first endoscopy unit via a fixing ring. When the multi-stage endoscopy module moves to the region near the target observing position, the control module releases the fixing ring to make the second endoscopy unit extending out of the first endoscopy unit and controls the second endoscopy unit to generate slight deformation to observe the real-time state of the target observing position through the movement or rotation of the second endoscopy unit.

Compared to the prior arts, the invention discloses the endoscopy apparatus having high degree of motion freedom and operating method thereof not only can maintain the advantage of the conventional endoscopy to smoothly move and test in human body or organ, but also can control the soft endoscopy unit to generate slight deformation to perform a detail test or sampling on the target observing position through a movement or rotation.

Therefore, the endoscopy apparatus having high degree of motion freedom and the operating method thereof disclosed by the invention can overcome the problems that the current endoscopy encountered clinically, and can be used to perform more detail test, sampling, or treatment on the organs in patient's body. Therefore, the quality of medical care can be enhanced and the health and the right of the patient can be also ensured accordingly.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An endoscopy apparatus having high degree of motion freedom, comprising:
a multi-stage endoscopy module, comprising at least a first endoscopy unit and a second endoscopy unit, the first endoscopy unit and the second endoscopy unit providing a first bending and a second bending respectively, wherein the second bending is larger than the first bending; and
a control module, when the multi-stage endoscopy module moves to a region near a target observing position, the control module controlling the second endoscopy unit to generate slight deformation to observe a real-time state of the target observing position.

2. The endoscopy apparatus of claim 1, wherein an end of the multi-stage endoscopy module is composed of the second endoscopy unit, and the other parts of the multi-stage endoscopy module except the end are composed of the first endoscopy unit, the multi-stage endoscopy module controls the movement or rotation of the second endoscopy unit on the end to observe the real-time state of the target observing position.

3. The endoscopy apparatus of claim 1, wherein in the multi-stage endoscopy module, the second endoscopy unit is encapsulated within the first endoscopy unit, when the multi-stage endoscopy module moves to the region near the target observing position, the control module controls the second endoscopy unit extending out of the first endoscopy unit and controls the second endoscopy unit to generate slight deformation to observe the real-time state of the target observing position through the movement or rotation of the second endoscopy unit.

4. The endoscopy apparatus of claim 1, wherein in the multi-stage endoscopy module, the second endoscopy unit is encapsulated within the first endoscopy unit, when the multi-stage endoscopy module moves to the region near the target observing position, the control module controls the first endoscopy unit to shrink to expose the second endoscopy unit out of the first endoscopy unit and controls the second endoscopy unit to generate slight deformation to observe the real-time state of the target observing position through the movement or rotation of the second endoscopy unit.

5. A method of operating an endoscopy apparatus having high degree of motion freedom, the endoscopy apparatus having high degree of motion freedom comprising a multi-stage endoscopy module and a control module, the multi-stage endoscopy module comprising at least a first endoscopy unit and a second endoscopy unit, the first endoscopy unit and the second endoscopy unit providing a first bending and a second bending respectively, wherein the second bending is larger than the first bending, the method comprising steps of:
(a) the multi-stage endoscopy module moving to a region near a target observing position;
(b) the control module controlling the second endoscopy unit to generate slight deformation; and
(c) the second endoscopy unit observing a real-time state of the target observing position.

6. The method of claim 5, wherein an end of the multi-stage endoscopy module is composed of the second endoscopy unit, and the other parts of the multi-stage endoscopy module except the end are composed of the first endoscopy unit, the multi-stage endoscopy module controls the movement or rotation of the second endoscopy unit on the end to observe the real-time state of the target observing position.

7. The method of claim 5, wherein in the multi-stage endoscopy module, the second endoscopy unit is encapsulated within the first endoscopy unit, when the multi-stage endoscopy module moves to the region near the target observing position, the control module controls the second endoscopy unit extending out of the first endoscopy unit and controls the second endoscopy unit to generate slight deformation to observe the real-time state of the target observing position through the movement or rotation of the second endoscopy unit.

8. The method of claim 5, wherein in the multi-stage endoscopy module, the second endoscopy unit is encapsulated within the first endoscopy unit, when the multi-stage endoscopy module moves to the region near the target observing position, the control module controls the first endoscopy unit to shrink to expose the second endoscopy unit out of the first endoscopy unit and controls the second endoscopy unit to generate slight deformation to observe the real-time state of the target observing position through the movement or rotation of the second endoscopy unit.
